(19) United States
(12) Patent Application Publication
(10) Pub. No.: US 2024/0341724 A1
(43) Pub. Date: Oct. 17, 2024

(54) MEDICAL IMAGING DEVICES

(71) Applicant: Boston Scientific Scimed, Inc., Maple Grove, MN (US)

(72) Inventors: Andrew Brian Graveley, Shoreview, MN (US); Daniel J. Foster, Lino Lakes, MN (US); Sebastian Ordas Carboni, Vadnais Heights, MN (US); Kyle True, Minneapolis, MN (US); Lawrence Jay Backman, Shoreview, MN (US)

(21) Appl. No.: 18/634,758

(22) Filed: Apr. 12, 2024

Related U.S. Application Data
(60) Provisional application No. 63/495,969, filed on Apr. 13, 2023.

(51) Int. Cl. A61B 8/12; A61B 5/06; A61B 8/00; A61B 8/08

(52) U.S. Cl. CPC A61B 8/12; A61B 5/062; A61B 8/0883; A61B 8/4488

(57) ABSTRACT

A medical imaging device is disclosed. The medical imaging device includes an ultrasound transducer having an acoustic stack comprising an active surface opposite a backing surface, a magnetic tracking sensor assembly, and a layered circuit assembly electrically and mechanically coupled to the ultrasound transducer and the magnetic tracking sensor assembly. The magnetic tracking sensor assembly includes a coupling surface. The backing surface aligns with the coupling surface.

### CROSS REFERENCE TO RELATED APPLICATIONS

This application claims the benefit of U.S. Provisional Application No. 63/495,969 entitled “MEDICAL IMAGING DEVICES,” filed Apr. 13, 2023, which is incorporated by reference herein in its entirety.

### TECHNICAL FIELD

The present disclosure relates to medical systems and methods for facilitating visualization of tissue from within a patient. More specifically, the present disclosure relates to medical systems, catheters, and methods having ultrasonic transducer devices.

### BACKGROUND

Intracardiac echocardiography (ICE) is an ultrasonic imaging modality that has become a common aspect of several percutaneous interventional and electrophysiology procedures. ICE can provide high resolution and real-time visualizations of cardiac structures, continuous monitoring of catheter location within the heart, and early recognition of procedural complications such as pericardial effusion or thrombus formation. An ICE catheter is inserted in a vein or artery, such as via standard femoral venous introducer into the femoral vein, and then passed from the vein or artery into the heart alongside other tools without fluoroscopy. Another common ultrasonic imaging modality includes transesophageal echocardiography (TEE) and fluoroscopy to image and visualize tools within the heart, which requires an anesthesiologist and additional echocardiologist to keep the patient under general anesthesia and to manipulate an ultrasonic probe within the airway. In contrast to TEE, ICE can be performed by the primary operator of the interventional procedure under conscious sedation, without endotracheal intubation, and with a reduced risk of esophageal trauma. Additionally, ICE reduces fluoroscopy exposure for both the patient and the operator. For these reasons, ICE holds promise to replace TEE as the preferred imaging modality in certain procedures such as atrial septal defect closure and catheter ablation of cardiac arrhythmias and includes an emerging role in other procedures such as mitral valvuloplasty, transcatheter aortic valve replacement, and left atrial appendage closure.

Two forms of ICE are available. Radial or rotational ICE uses a single piezoelectric crystal mounted on a tip of a six to ten French catheter. A rotating transducer provides cross-sectional images in a radial plane perpendicular to a longitudinal axis of the catheter. Rotational ICE operates at imaging frequencies that are useful for near-field imaging of up to six or eight centimeters but is limited for far-field imaging. Phased-array ICE uses a multi-element transducer, such as a sixty-four-element transducer, mounted on a distal end of an eight to ten French steerable catheter that can often be deflected in four directions including anterior, posterior, right, and left. The device provides a wedge-shaped image, such as a ninety-degree sector plane, from the side of the catheter that can be displayed on a conventional ultrasound workstation. When contrasted with mechanical rotational ICE systems, phased-array ICE can provide for greater image-depth penetration (up to 15 cm), greater maneuverability, and the ability to acquire Doppler and color flow imaging.

### SUMMARY

In Example 1, a medical imaging device comprising an ultrasound transducer having an acoustic stack comprising an active surface opposite a backing surface, a magnetic tracking sensor assembly, and a layered circuit assembly electrically and mechanically coupled to the ultrasound transducer and the magnetic tracking sensor assembly, the magnetic tracking sensor assembly having a coupling surface, wherein the backing surface aligns with the coupling surface.

In Example 2, the medical imaging device of Example 1, wherein the layered circuit assembly includes a flexible circuit.

In Example 3, the medical imaging device of any of Examples 1-2, wherein the flexible circuit is generally planar and includes a first portion and a second portion, wherein the magnetic tracking sensor assembly and ultrasound transducer are electrically coupled to the first portion, and the second portion is folded underneath the first portion.

In Example 4, the medical imaging device of any of Examples 1-3, wherein the magnetic tracking sensor assembly is disposed within an encapsulant.

In Example 5, the medical imaging device of Example 4, wherein the encapsulant is an epoxy.

In Example 6, the medical imaging device of any of Examples 1-5, wherein the magnetic tracking sensor assembly includes one of a plurality of tunneling magneto-resistive sensors and a plurality of inductive sensors.

In Example 7, the medical imaging device of any of Examples 1-6, wherein the ultrasound transducer includes a matching layer, wherein the matching layer includes the active surface.

In Example 8, the medical imaging device of any of Examples 1-7, wherein the ultrasound transducer includes a backing layer, the backing layer having the backing surface, wherein the backing surface includes a plurality of grooves in the backing layer.

In Example 9, the medical imaging device of any of Examples 1-8, wherein the ultrasound transducer includes a phased array transducer.

In Example 10, the medical imaging device of any of Examples 1-9, wherein the medical imaging device is included in a distal cover attached to a catheter shaft.

In Example 11, the medical imaging device of Example 10, wherein the catheter is included in an imaging system further comprising a plurality of magnetic field transmitter assemblies.

In Example 12, the medical imaging device of Example 11, wherein the imaging system further includes a controller operably coupled to the medical imaging device, the controller configured to receive a sensing signal from the ultrasonic transducer and a tracking signal from the magnetic tracking sensor assembly and generate an image including location and orientation of a two-dimensional imaging plane in a three-dimensional space.

In Example 13, the medical imaging device of Example 1, wherein the magnetic tracking assembly is attached to the layered circuit assembly via a wirebonding process.

In Example 14, the medical imaging device of Example 12, wherein the layered circuit assembly is folded into a stack, the stack disposed on the magnetic tracking assembly.

In Example 15, the medical imaging device of any of Examples 1-14, wherein the ultrasound transducer is configured as a two-dimensional imaging phased array assembly, which include a one-dimensional device.

In Example 16, a medical imaging device comprising an ultrasound transducer having an acoustic stack comprising an active surface opposite a backing surface, a magnetic tracking sensor assembly, and a layered circuit assembly electrically and mechanically coupled to the ultrasound transducer and the magnetic tracking sensor assembly, the magnetic tracking sensor assembly having a coupling surface, wherein the backing surface aligns with the coupling surface.

In Example 17, the medical imaging device of Example 16, wherein the layered circuit assembly includes a flexible circuit.

In Example 18, the medical imaging device of Example 17, wherein the flexible circuit includes a first portion coupled to a folded stack, wherein the magnetic tracking sensor assembly and ultrasound transducer are electrically coupled to the first portion, and the foldable stack is folded underneath the first portion.

In Example 19, the medical imaging device of Example 18, wherein the flexible circuit includes a plurality of sets of longitudinally extending conductive elements disposed between longitudinally extending perforations.

In Example 20, the medical imaging device of Example 18, wherein the ultrasound transducer and the magnetic tracking sensor assembly each include a plurality of electrical connections electrically coupled to the longitudinally extending conductive elements.

In Example 21, the medical imaging device of Example 16, wherein the magnetic tracking sensor assembly is disposed within an encapsulant.

In Example 22, the medical imaging device of Example 21, wherein the encapsulant is an epoxy.

In Example 23, the medical imaging device of Example 16, wherein the magnetic tracking sensor assembly includes one of a plurality of tunneling magneto-resistive sensors and a plurality of inductive sensors.

In Example 24, the medical imaging device of Example 23, wherein the plurality of inductive sensors each include longitudinal portion angled toward each other.

In Example 25, the medical imaging device of Example 23, wherein the magnetic tracking sensor assembly includes the plurality of tunneling magneto-resistive sensors electrically coupled to corresponding sensor circuits.

In Example 26, the medical imaging device of Example 16, wherein the ultrasound transducer includes a matching layer, wherein the matching layer includes the active surface.

In Example 27, the medical imaging device of Example 16, wherein the ultrasound transducer is configured as a two-dimensional imaging phased array assembly, which include a one-dimensional device.

In Example 28, the medical imaging device of Example 16, wherein the ultrasound transducer includes a backing layer, the backing layer having the backing surface, wherein the backing surface includes a plurality of grooves in the backing layer.

In Example 29, a medical imaging system comprising a magnetic field transmitter assembly configured to generate a magnetic field; a medical imaging catheter having a distal end region including a medical imaging module, the medical imaging module comprising an ultrasound transducer having an acoustic stack comprising an active surface opposite a backing surface, the ultrasound transducer configured to transform acoustic energy received by the acoustic stack and to provide a corresponding sensing signal, a magnetic tracking sensor assembly, the magnetic tracking assembly configured to sense the generated magnetic field and to provide a corresponding tracking signal associated with a location of the magnetic tracking sensor assembly, and a layered circuit assembly electrically and mechanically coupled to the ultrasound transducer and the magnetic tracking sensor assembly, the magnetic tracking sensor assembly having a coupling surface, and wherein the backing surface aligns with the coupling surface; and a controller operably coupled to the medical imaging module, the controller configured to receive the sensing signal and the tracking signal and to generate an image including location of two-dimensional imaging plane in a three-dimensional space.

In Example 30, the medical imaging system of Example 29, wherein the controller is further configured to generate a three-dimensional map of the heart.

In Example 31, the medical imaging system of Example 29, wherein the tracking signal further provides orientation of the magnetic tracking sensor assembly and the generated image includes the location and an orientation of the two-dimensional imaging plane.

In Example 32, the medical imaging device of Example 29, wherein the ultrasound transducer is configured as a two-dimensional imaging phased array assembly having a one-dimensional array device to provide two-dimensional images by steering an acoustic beam across a two-dimensional plane.

In Example 33, a method for manufacturing a medical imaging device, the method comprising electrically coupling a magnetic tracking sensor assembly to a first portion of a flexible circuit; folding a remaining portion of the flexible circuit into a flexible circuit stack; and electrically coupling an ultrasound transducer to the flexible circuit, wherein the flexible circuit includes a first side of the first portion interfacing with the backing surface and an opposite, second side of the first portion interfacing with the magnetic tracking sensor assembly.

In Example 34, the method of Example 33, wherein the flexible circuit stack is disposed on the magnetic tracking sensor assembly.

In Example 35, the method of Example 34, wherein the magnetic tracking sensor assembly is encapsulated in an epoxy prior to folding the flexible circuit into the flexible circuit stack.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using a medical imaging system, in accordance with embodiments of the subject matter of the disclosure.

FIG. 2 is a perspective view of a medical imaging device that can be used with the example medical imaging system of FIG. 1.

FIG. 3 is a schematic diagram illustrating a feature of the medical imaging device of FIG. 2.

FIG. 4A is a schematic diagram illustrating a perspective view of an example of the feature of the medical imaging device of FIG. 3.

FIG. 4B is a schematic diagram illustrating a front view of the example of the feature of the medical imaging device of FIG. 4A.

FIG. 4C is a schematic diagram illustrating a side view of the example of the feature of the medical imaging device of FIG. 4A.

FIG. 5 is a schematic diagram illustrating a component of the example of the feature of the medical imaging device of FIG. 4A.

FIG. 6 is a schematic diagram illustrating a perspective view of another example of the feature of the medical imaging device of FIG. 3.

FIG. 7 is a schematic diagram illustrating a component of the example of the feature of the medical imaging device of FIG. 6A.

FIG. 8 is a block diagram illustrating an example method of manufacturing the medical imaging device of FIG. 3.

FIG. 9 is a schematic diagram illustrating a component of the example of the feature of the medical imaging device of FIG. 4A as a feature of the method of manufacturing the medical imaging device of FIG. 8.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

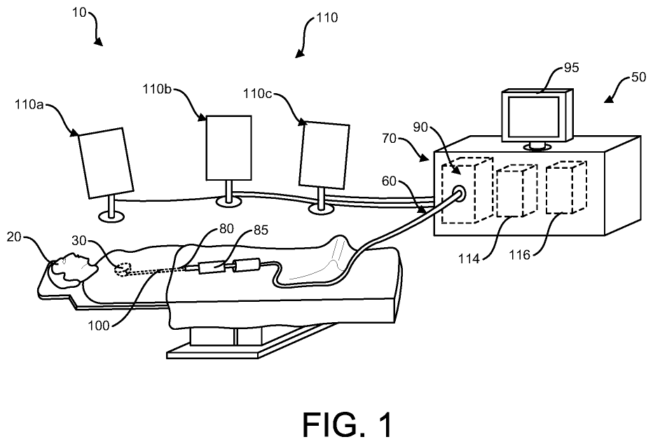

FIG. 1 illustrates an example clinical setting 10 for a medical procedure on a patient 20, such as a medical procedure on a heart 30 of the patient 20, using a medical imaging system 50 in accordance with the disclosure. The medical imaging system 50 includes a medical imaging catheter system 60 configured to be inserted into the patient 20 for imaging of portions of the patient's anatomy. The medical imaging catheter system 60 includes a medical imaging catheter 80, and introducer sheath 85, and an imaging console 90. Additionally, the medical imaging catheter system 60 includes various connecting elements, such as cables and tubing, that operably connect the components of the medical imaging catheter system 60 to one another and to the components of the medical imaging system 50. In the example, the introducer sheath 85 is operable to provide a delivery conduit through which the medical imaging catheter 80 can be deployed to the specific target sites within the patient's heart 30. Access to the patient's heart 30 can be obtained through a vessel, such as a peripheral artery or vein often in the groin or possibly in the shoulder or neck. Once access to the vessel is obtained, the medical imaging catheter 80 can be navigated to within the patient's heart 30, such as within a chamber of the heart.

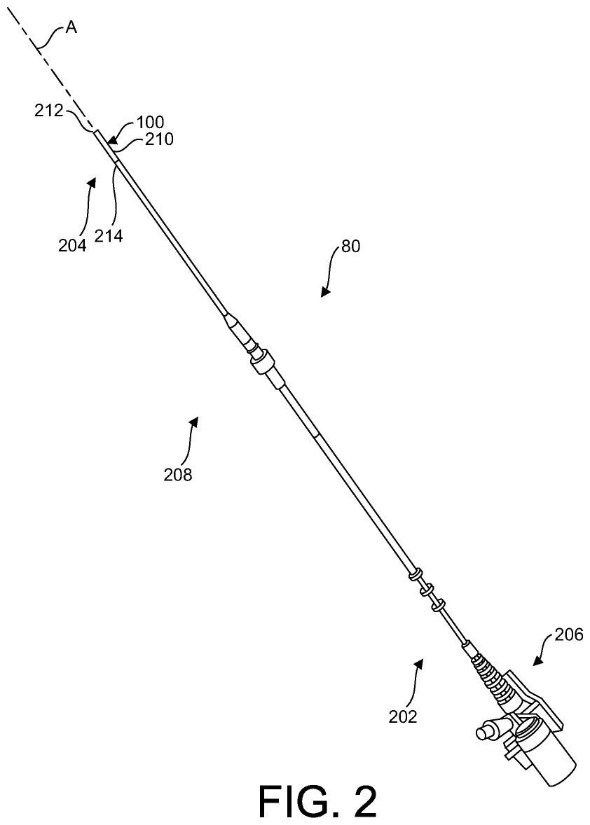

FIG. 2 illustrates an example medical imaging catheter 80 suitable for use with the medical imaging catheter system 60. The medical imaging catheter 80 includes a proximal end region 202 and a distal end region 204. The medical imaging catheter 80 defines a longitudinal axis A that passes through a centroid of a cross section of the distal end region 204. The proximal end region 202 of the medical imaging catheter 80 includes a catheter hub 206, and the distal end region 204 is configured and arranged to be inserted into the patient 20. The medical imaging catheter 80 includes a shaft 208 that is dimensioned to be inserted within regions of the patient 20 that are difficult to navigate, includes blood vessels, heart chambers, the gastrointestinal tract, the urinary tract, and the pulmonary system. The distal end region 204 includes an imaging module 100 that can be, for example, extended into the patient's heart 30 and disposed at the end of the shaft 208. The proximal end region 202 is configured to be coupled to the medical imaging console 90. The imaging module 100 can be electrically coupled to elongated lead conductors that extend from the distal end region 204 along the catheter hub at the proximal end region 202. The lead conductors can be insulated from one another within an insulating sheath, such as with an insulating polymer sheath extending the length of the shaft 208. The lead conductors can be electrically coupled to a plug in the proximal end region 202, such as a plug configured to be mechanically and electrically coupled to the medical imaging console 90, for example, either directly or via intermediary electrical conductors such as cabling. In the example, the imaging module 100 can include an imaging module cover 210 forming a distalmost tip 212, the imaging module cover 210 being mechanically coupled to a distal end 214 of the shaft 208.

Returning to FIG. 1, the medical imaging console 90 includes a controller, such one or more controllers, processors, or computers, that executes instructions or code, such as processor-executable instructions, stored on a non-transitory computer readable medium, such as a memory device, or memory, to cause, such as control or perform, the aspects of the medical imaging catheter system 60. The medical imaging console 90, in one example, can include a display 95. In one example, the medical imaging console 90 is configured to provide an electrical signal, such as a plurality of concurrent or space-apart-time electrical signals, to the electrically connected medical imaging catheter 80 along lead conductors to the imaging module 100. The medical imaging console 90 is also configured to receive an electrical signal, such as a plurality of concurrent or space-apart-time electrical signals, from the electrically connected medical imaging catheter 80 along lead conductors from the imaging module 100. The imaging module 100 includes an ultrasound imaging device. The medical imaging catheter system 60 is configured to transmit and receive acoustic energy to generate ultrasound images.

In one example, the ultrasound imaging device is configured as an intracardiac echocardiography (ICE) device. In other examples, the ultrasound imaging device can be configured as an endobronchial ultrasound (EBUS) device, an intra-vascular ultrasound (IVUS) device, or any other type of ultrasound imaging device. The ultrasound imaging device provides a wedge-shaped image, such as a ninety-degree sector plane, from the side of the distal end of the medical imaging catheter 80 that can be displayed on the medical imaging console 90. In one example of the ultrasound imaging device, a two-dimensional imaging phased array device, which include one-dimensional arrays, creates two-dimensional images by steering an acoustic beam across a two-dimensional plane. Each acoustic beam, or scan line, produces echoes that are measured and combined with other scan lines into an ultrasound image. To create a three-dimensional or four-dimensional (including time as a dimension) image, a two-dimensional array is applied to steer the beam throughout a three-dimensional volume or two orthogonally disposed one-dimensional arrays scan each scan beams across a respective two-dimensional plane. Due to the physics of beamforming, exponentially more elements, interconnects and powering circuits are used, which includes increased costs as well as leads to increased dimensions of the distal end of the medical imaging catheter. In many examples, the increased sizes of four-dimensional ICE transducers are much less maneuverable through tortious anatomy and are more tedious to access many areas of the heart for closer imaging. Even if the increased costs and sizes can be tolerated, a medical imaging catheter system still produces a wedge-shaped image, such as a ninety-degree sector plane, which is plus/minus forty-five degrees in the axis perpendicular to the longitudinal axis of the distal end of the medical imaging catheter for a side-firing ICE device. In such a configuration, a typical medical imaging catheter system is not able to generate an image of what is in front of, i.e., in the direction of the longitudinal axis beyond the distal end, of the medical imaging catheter.

To create a three-dimensional or four-dimensional image with the medical imaging system 50 of the present disclosure, the imaging module 100 includes a phased array ultrasound transducer, such as a one-dimensional phased array transducer in combination with a magnetic tracking sensor assembly, and the medical imaging system 50 includes a magnetic tracking system 70, such as a magnetic tracking system 70 incorporated with medical imaging catheter system 60. As included in the imaging module 100, the one-dimensional phased array ultrasound transducer provides signals for a two-dimensional image on a single plane, and the magnetic tracking sensor provides signals for position and orientation in six degrees of freedom. With the imaging module 100, the medical imaging system 50 can produce provide a two-dimensional imaging plane in which the plane is located and oriented in a three-dimensional space. The medical imaging system 50 can allow a user to move and rotate the distal end region 104 to scan and image a three-dimensional volume. Additionally, images generated with the medical imaging system 50 can be fused with computed tomography (CT) images and magnetic resonance images (MRIs) to provide additional information during a procedure. The phased array ultrasound transducer, such as a one-dimensional phased array one-dimensional transducer, in combination with a magnetic tracking sensor assembly are disposed within the imaging module cover 208 attached to the shaft 208 of the medical imaging catheter 80.

As illustrated in FIG. 1, the magnetic tracking system 70 includes a magnetic field transmitter assembly 110, such as magnetic field transmitter assemblies 110a, 110b, . . . 110n, a magnetic field controller 114, and a signal processor 116. The magnetic field transmitter assemblies 110a, 110b, . . . 110n, illustrated schematically, are configured to transmit or radiate electromagnetic signals, which produce a magnetic field within which a patient 20 is disposed. The magnetic field controller 114 is configured to manage operation of the magnetic field transmitter assembly 110. For instance, the magnetic field controller 114 includes a signal generator configured to provide a driving current, such as a variable driving current, to each magnetic field transmitter assembly 110a, 110b, . . . 110n, to cause each magnetic field transmitter assembly 110 to transmit an electromagnetic field. In one example, at least one transmitter assembly is near the magnetic tracking assembly module, such as underneath the patient 20 (or underneath the patient's bed), to increase signal to noise ratio. The magnetic tracking system 70, such as the magnetic field controller 114 and signal processor 116, includes a controller, such one or more controllers, processors, or computers, that executes instructions or code, such as processor-executable instructions, stored on a non-transitory computer readable medium, such as a memory device, or memory, to cause, such as control or perform, the aspects of the magnetic tracking system 70.

The magnetic tracking sensor assembly of the imaging module 100, in the example, is configured to produce an electrical response to the magnetic field or magnetic fields generated by the magnetic field transmitter assembly 110. For example, the magnetic tracking sensor assembly includes a magnetic field sensor such as an inductive sensing coil or various sensing elements such as a magneto-resistive (MR) sensing element—which can include, for instance, anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, and spin Hall sensing elements—a giant magneto-impedance (GMI) sensing element, or flux-gate sensing element. In one example, the magnetic tracking sensor assembly includes a plurality of magnetic field sensors.

The magnetic tracking sensor assembly of the imaging module 100 is configured to sense the generated magnetic fields and provide a tracking signal indicating the location and orientation of the magnetic tracking sensor assembly, or imaging module 100, in up to six degrees of freedom (i.e., x-, y-, and z-axis translation, and pitch, yaw, and roll rotation). Generally, the number of degrees of freedom that a tracking system can track depends on the number of magnetic field sensors and magnetic field generators. For example, a tracking system with a single magnetic field sensor may not be capable of tracking roll angles and thus are limited to tracking in only five degrees of freedom (i.e., x, y, and z coordinates, and pitch and yaw angles). This is because a magnetic field sensed by a single magnetic field sensor does not change as the single magnetic field sensor is “rolled.” The magnetic field sensors can be powered by voltages or currents to drive or excite elements of the magnetic field sensors. The magnetic tracking sensor assembly of the imaging module 100 is communicatively coupled to the signal processor 116 such as via leads to the imaging module 100 electrically coupled to the signal processor 116. The magnetic field sensors receive the voltage or current and, in response to one or more of the generated magnetic fields, the magnetic field sensors generate the tracking signal, which is provided to the signal processor 116.

The tracking signal may include multiple magnetic field signals, each of which may be processed to extract field components corresponding to one or more magnetic field transmitter assemblies 110a, 110b, . . . , 110n. The tracking signal is communicated to the signal processor 116, which is configured to analyze the sensed magnetic field signal to determine location or location and orientation information corresponding to the magnetic tracking sensor assembly of the imaging module 100. Location information may include any type of information associated with a location or position of the imaging module 100 such as, for example, location, relative location (e.g., location relative to another device), position, orientation, velocity, and acceleration. In some examples, the signal processor 116 can also be communicatively coupled to a reference magnetic sensor assembly to detect the magnetic fields generated by the magnetic field transmitter assembly 110, such as a reference sensor coupled to the patient 20, or the bed, and in some examples a reference sensor can be coupled to the magnetic field transmitter assembly 110 and communicatively coupled to the signal processor 116.

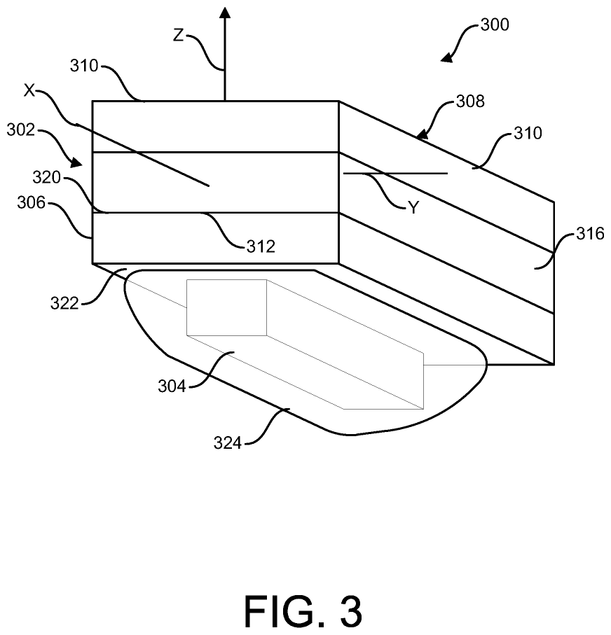

FIG. 3 illustrates an example medical imaging device 300 corresponding with the imaging module 100 described above. The medical imaging device 300 includes an ultrasound transducer 302, a magnetic tracking sensor assembly 304, and a layered circuit assembly 306 electrically and mechanically coupled to the ultrasound transducer 302 and the magnetic tracking sensor assembly 304. The magnetic tracking assembly includes a coupling surface. In the example, the medical imaging device 300 includes a longitudinal axis X that can correspond and be coaxial with longitudinal axis A in the case of the medical imaging device disposed on the distal end region 204 of medical imaging catheter 80. The ultrasound transducer 302 and the magnetic tracking assembly 304 are coupled to the layered circuit assembly 306 such that the ultrasound transducer 302 and the magnetic tracking assembly 304 are aligned, such as aligned along the axis orthogonal to the longitudinal axis A. In various examples, the circuit assembly 306 is one or more of a flexible printed circuit board or an integrated circuit (such as an ASIC).

The ultrasound transducer 302 includes an acoustic stack 308 comprising a plurality of layers forming an active surface or active side 310 opposite a backing surface or backing side 312. For example, the acoustic stack is developed as layers along a vertical axis Z generally perpendicular to the longitudinal axis X in a radial direction. In one example, the ultrasound transducer 302 includes an active layer 314 disposed against a backing layer 316. Active layer 314 may be formed from one or more known materials capable of transforming applied electrical signals to acoustic energy emanating from a surface of the active layer 314, such as an active side, and conversely capable of transforming acoustic energy absorbed by the active layer 314 into electrical signals. Examples of suitable materials for the active layer 314 include piezoelectric ceramic materials, piezocomposite materials, piezoelectric plastics, barium titanates, lead zirconate titanates, lead metaniobates, and polyvinylidenefluorides. Other transducer technologies may include composite materials, single-crystal composites, and semiconductor devices, such as capacitive micromachined ultrasound transducers (“cMUT”) and piezoelectric micromachined ultrasound transducers (“pMUT”). Backing layer 316 can be formed from materials suitable for absorbing, scattering, or attenuating acoustic energy, including polymers, composite materials incorporating metallic scattering particles or ceramic oxide scattering particles, and viscoelastic materials. Backing layer 316 dampens vibration of active layer 314 to control the pulse length and pulse duration or vibration of the active layer 314 when fired. When positioned adjacent to the active layer 314, backing layer 316 absorbs, scatters, or attenuates acoustic energy emitted from active layer 314 to cease vibrating rapidly when application of the electrical signal ceases. In some examples, the acoustic stack 308 can include a matching layer, such as a plurality of matching layers, disposed on the active layer 314 to form the active side. Matching layers are used in acoustic stacks of ultrasound transducers, for example, to facilitate the transfer of acoustic energy from the active layer to the propagating medium and vice versa within a bandwidth of frequencies around the center frequency of the device.

In one example, the ultrasound transducer 302 is configured as a two-dimensional imaging phased array assembly, which include one-dimensional arrays to create two-dimensional images by steering an acoustic beam across a two-dimensional plane. Other configurations of the ultrasound transducer 302 are contemplated, and the ultrasound transducer 302 can include linear arrays, curvilinear arrays, 1.25-dimensional phased arrays, 1.5-dimensional phased arrays, 1.75-dimensional phased arrays, or two-dimensional arrays. In one example, the ultrasound transducer 302 is configured as a side firing ultrasound transducer in the medical imaging device 300 and in an imaging module 100 of the medical imaging catheter 80. Other configurations of the medical device 300 are contemplated, such as a front facing transducer, radial transducer, or a combination.

The magnetic tracking sensor assembly 304 includes a plurality of magnetic field sensors and corresponding sensor circuits. For example, the magnetic tracking sensor assembly 304 includes first, second, and third magnetic field sensors and first, second, and third sensor circuits. Each sensor circuit can include circuits with a diode and capacitor. The magnetic field sensors and the sensor circuits can be implemented on separate dies and disposed next to each other or near each other. The magnetic field sensors and the sensor circuits can be electrically coupled together. In some examples, respective magnetic field sensors and the corresponding sensor circuits can be implemented on the same die or substrate such as in a monolithic design. For example, the magnetic field sensor can be fabricated on top of the corresponding sensor circuit. magnetic field sensor such as an inductive sensing coil or various sensing elements such as an MR sensing element--which can include, for instance, AMR sensing elements, GMR sensing elements, TMR sensing elements, Hall effect sensing elements, CMR sensing elements, EMR sensing elements, and spin Hall sensing elements—a GMI sensing element, or flux-gate sensing element. The magnetic field sensors are configured to sense magnetic fields, such as magnetic fields generated by the magnetic field transmitter assembly 110, and generate a responsive sensing signals.

The magnetic field sensors can be positioned in a dual-axis, six degree-of-freedom arrangement. For example, the first magnetic field sensor and the second magnetic field sensor are oriented such that their primary sensing direction is aligned along the longitudinal axis X of the medical imaging device 300. The third magnetic field sensor is oriented such that its primary sensing direction is aligned along an axis orthogonal to the longitudinal axis X. In some examples, the magnetic field sensors are positioned in a tri-axis, six degree-of-freedom arrangement. In such examples, the magnetic field sensors' primary sensing directions are orthogonal to each other. In certain examples, one or more of the magnetic field sensors is a dual-axis or tri-axis sensor having two or three primary sensing directions, respectively. The first, second, and third magnetic field sensors are configured to generate, in response to a magnetic field, responsive sensing signals. The sensing signals are used to determine location and orientation of the sensor assembly 300.

The layered circuit assembly 306 includes a first side 320 interfacing with the backing side 312 of the ultrasound transducer 302, and the layered circuit assembly 306 includes an opposite, second side 322 interfacing with the magnetic tracking sensor assembly 304. In the examples, the layered circuit assembly 306 can include a printed circuit or flex circuit electrically coupled to the ultrasound transducer 302 and the magnetic tracking sensor assembly 304. In the example, the magnetic tracking sensor assembly 304 includes a cover 324, such as an epoxy encapsulant for rigidity. The first side 320 and second side 322 can be on opposite surfaces of the layered circuit assembly 306, or, in the case of a layered circuit assembly folded onto itself (or folded an odd number of times onto itself), the first side 320 and second side 322 can be on the same surface of an unfolded layered circuit assembly 306 such that the ultrasound transducer 302 and the magnetic tracking sensor assembly 304 are on opposite sides of the layered circuit assembly 306. The layered circuit assembly 306 can be folded multiple times to form a folded stack circuit assembly, such as between the ultrasound transducer 302 and the magnetic tracking sensor assembly 304 or tucked underneath the magnetic sensor assembly 304 and stacked along the vertical axis Z.

The ultrasound transducer 302 includes electrical connectors that are attached to mounting pads of the layered circuit assembly 306. Electrical signals may be applied to the active layer 314 via the electrical connecters 306. For example, a voltage may be applied to active layer 304 via electrical connecters the flex circuit of the layered circuit assembly 306 to generate a pulse of acoustic energy that emanates from active layer 314 toward an imaging target. The pulse of acoustic energy may include one or more acoustic waves and may have an associated pulse duration and pulse length. When the pulse of acoustic energy reflects off the imaging target and the reflection is received by active layer 314, active layer 314 may generate an electrical signal that may be measured or detected and provided from the medical imaging device via electrical connecters 306. The electrical signal may in turn be used to generate an ultrasound image of the target via the medical imaging system 50. Similarly, the magnetic tracking sensor assembly 304 includes electrical connectors that are attached to mounting pads on the second side 322 of the layered circuit assembly 306 to receive and provide the sensing signals to the medical imaging system 50 to determine location and orientation of the medical imaging device 300.

As provided from the medical imaging device 300, the sensing signals from the ultrasound image are combined with the tracking signals in the medical imaging system 50, for example, via a controller to generate both a two-dimensional imaging plane as well as the location or the location and orientation of the two-dimensional imaging plane in three-dimensional space for an image. The medical imaging device 300 can be applied with a tracking system and software program run by the medical imaging system 50 to allow for movement and rotation to scan and image a three-dimensional volume and construct three-dimensional images. Additionally, the medical device 300 can enable image fusion with CT or MRI images to provide for additional information during a procedure. The medical device 300 addresses previous issues with two-dimensional and four-dimensional TEE and ICE devices by generating signals to provide information to generate two-dimensional and four-dimensional images and maps to guide clinicians through procedures in comparable costs and sizes of two-dimensional imaging ICE devices. For instance, an ultrasound transducer 302 configured from a one-dimensional phased array transducer applied in the medical imaging device 300 can provide for robust imaging with low cost and small form factors. In one example, the imaging device can be provided on a nine French catheter. Also, an ultrasound transducer 302 configured from, for example, a two-dimensional phased array, for four-dimensional ultrasound imaging, applied in the medical imaging device 300 can provide for additional useful information in, for example, generating three-dimensional maps of anatomy, such as cardiac anatomy.

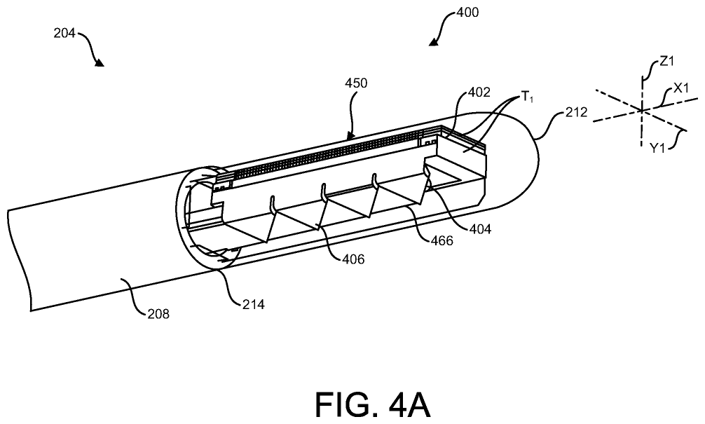

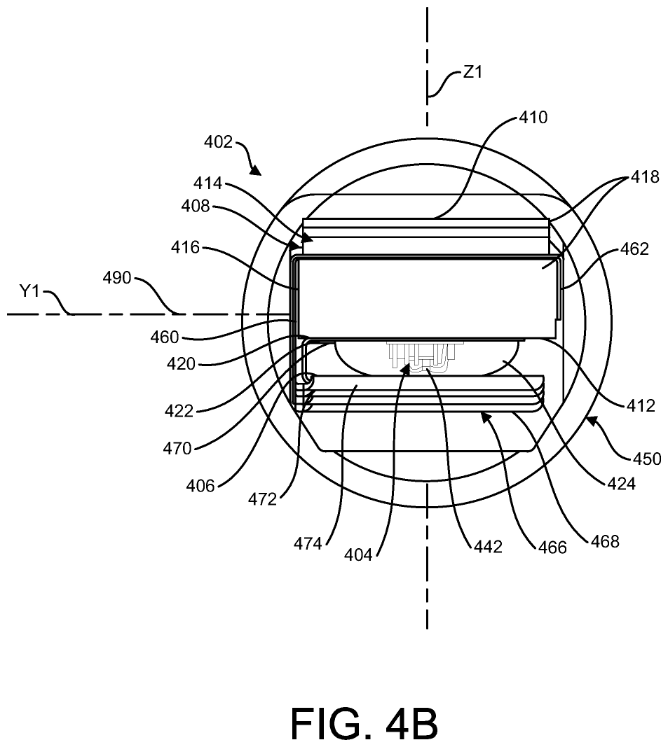

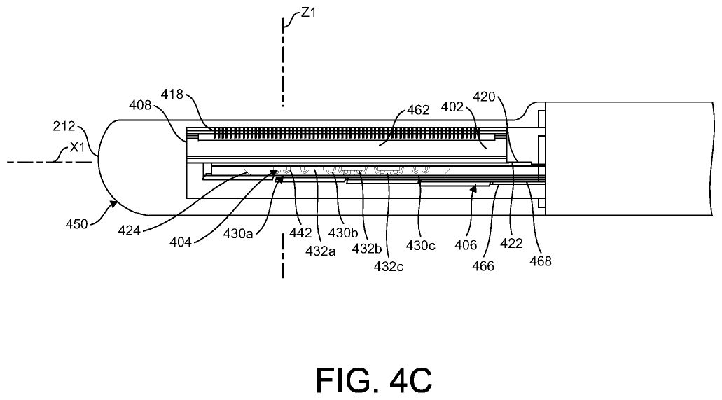

FIGS. 4A-4C illustrate an example medical imaging device 400 constructed in accordance with the medical imaging device 300. In the example, the medical imaging device 400 is installed on a medical imaging catheter 80. The medical imaging device 400 includes an ultrasound transducer 402, a magnetic tracking sensor assembly 404, and a flex circuit 406 electrically coupled to the ultrasound transducer 402 and the magnetic tracking sensor assembly 404. In the example, the medical imaging device 400 includes a longitudinal axis X1, and the longitudinal axis X1 can correspond to and be coaxial with longitudinal axis A in the case of the medical imaging device 400 disposed on the distal end region 204 of medical imaging catheter 80. In the example, the medical imaging device 400 can include, or in some examples be enclosed by, a distal tip cover 450 that is suitable for attachment to the distal end region 204 of the medical imaging catheter 80 such as abutted against the catheter shaft 208. In some examples, the distal tip cover 450 can be formed as a focusing lens.

The ultrasound transducer 402 is formed as an acoustic stack 408 having, for example, an active layer 414, a backing layer 416, and matching layers 418. The matching layer 418 provides an active side 410 of the acoustic stack 408, and the backing layer 416 provides a backing side 412 of the acoustic stack 408. The acoustic stack 408 is developed as layers along a vertical axis Z1 generally perpendicular to the longitudinal axis X1 in a radial direction. Length is measured along the longitudinal axis X1, and height is measured along the axis Z1. Width, such as width of the backing side 412, is measured along a lateral axis Y1 orthogonal to the longitudinal axis X1 and vertical axis Z1. In one example, the backing side 412 includes a plurality of spaced-apart grooves having a depth along vertical axis Z1 in the backing layer 416 to reduce thickness of the backing layer 416 and acoustic stack 408. Without being bound to a particular theory, the plurality of space-apart grooves can induce phase cancellation of acoustic energy as it reflects from the backing side 412 and returns to the active layer 414 to diminish reflection. Configurations of ultrasound transducers 402 and acoustic stacks 408 are illustrated and described in Andrew Graveley, et al., U.S. Patent Publication 2022/0409170A, titled ULTRASOUND TRANSDUCER and assigned to the present assignee, which is incorporated by reference in its entirety herein.

The magnetic tracking sensor assembly 404 of the example incorporates TMR sensors assembled directly to the flex circuit 406. For instance, the magnetic tracking sensor assembly 404 includes a dual-axis configuration having three magnetic field sensors 430a, 430b, 430, and corresponding sensor circuits 432a, 432b, 432c. For instance, the sensor circuits 432a, 432b, 432c can include a plurality of diodes and a capacitor. Configurations of the magnetic tracking sensor assembly 404 with magnetic field sensors 430a, 430b, 430c and sensor circuits 432a, 432b, 432c are illustrated and described in Steven J. Meyer, et al., U.S. Pat. No. 11,141,567, titled ELECTRICAL ARRANGEMENTS FOR SENSOR ASSEMBLIES IN ELECTROMAGNETIC NAVIGATION SYSTEMS and assigned to the present assignee, which is incorporated by reference in its entirety herein.

The flex circuit 406 includes a first side 420 interfacing with the backing side 412 of the ultrasound transducer 402, and the flex circuit 406 includes an opposite, second side 422 interfacing with the magnetic tracking sensor assembly 404. In the example, the first side 420 and second side 422 are on opposite surfaces of the flex circuit 406. The ultrasound transducer 402 includes electrical coupling members 440, which are operably coupled to the active layer 414 and extend along longitudinal sides 460, 462 of the ultrasound transducer 402, that are attached to the flex circuit on first side 420 of the flex circuit 406 such as on a first surface of the flex circuit 406. The magnetic tracking sensor assembly 404, particularly the magnetic field sensors 430a, 430b, 430c and sensor circuits 432a, 432b, 432c, includes electrical connectors 442 that are attached to the second side 422 of the flex circuit 406, such as on an opposite surface of the flex circuit 406. The flex circuit 406 includes a distal section 452 having a plurality of exposed electrical mounting pads 444 on a surface of the flex circuit 406. The electrical mounting pads 444 are conductively coupled via a plurality of conductive elements 448 to a plurality of electrical lead pads (not shown) on a proximal section 454 of the flex circuit 406. The electrical lead pads can be electrically coupled to leads within the catheter shaft 208 extending to the catheter proximal end 202 to provide electrical signals to connectors on the catheter proximal end 202.

In the case of the first side 420 and second side 422 on opposite surfaces of the flex circuit 406, one surface or both surfaces can include exposed electrical mounting pads 444. The magnetic tracking sensor assembly 404 is coupled to the second side 422 of the flex circuit 406 such as with electrical connectors 442 of the magnetic field sensors 430a, 430b, 430c and sensor circuits 432a, 432b, 432c attached to associated electrical mounting pads 444. The backing side 416 of the ultrasound transducer 402 can be disposed against or attached to the first side 420 of the flex circuit 406 such that the active side 414 of the acoustic stack is not facing the flex circuit 406, and the electrical connectors 440 of the ultrasound transducer 402 are electrically coupled to associated electrical mounting pads 444. In one example, the electrical coupling members and connectors 440, 442 are coupled to the associated electrical connectors via wire bonds or other suitable connections. Other examples include electrically coupling the ultrasound transducer 402 and magnet tracking sensor assembly via flip-chips, through-silicon vias, and fan-out wafer level packaging. In the example, the magnetic tacking sensor assembly 404, electrically coupled to the flex circuit 406 is covered with an epoxy encapsulant 424, disposed on the magnetic tracking sensor assembly 404 and the second side 422 of the flex circuit 406, such as for rigidity or protection of the components of the magnetic tracking sensor assembly 404.

In many examples, width of the flex circuit 406 with a plurality of laterally spaced-apart conductive elements 448 can be greater than multiples of the width of the acoustic stack 408 (such as measured along the width of the backing side 412) or the width of the magnetic tracking sensor assembly 404 positioned on the flex circuit 406. In such an example, the width of the medical imaging device 400 can be reduced by folding or rolling, or repeatedly folding or rolling, the flex circuit 406 to fit within the distal tip cover 450. In one example, the flex circuit 406 is folded into a flex circuit stack 466 such that width of the medical imaging device 400 is approximately the width of the backing side 412 or between the vertically extending electrical connectors 440 of the ultrasound transducer 402.

The examples illustrate the medical imaging device 400 having a flex circuit stack 466 disposed underneath the ultrasound transducer 402 and the magnetic tracking sensor assembly 404. The flex circuit 406 is folded into the flex circuit stack 466 to include a plurality of flex circuit layers 468 developed along the vertical axis Z1. The flex circuit 406 includes a first portion 470 including the first side 420 interfacing with the backing side 412 of the ultrasound transducer 402 and the second side 422 electrically coupled to the magnetic tracking sensor assembly 404 and including the encapsulant 424. The first portion 470 of the flex circuit 406 extends to a second, or vertical portion 472 that is folded generally perpendicularly to the first portion 470 having a height along the Z1 axis sufficient to clear the magnetic tracking sensor assembly 404 or the encapsulant 424 if included. The second portion 472 is folded from the first portion 470 to fit within the electrical connectors 440, such as within the width of the backing side 412. The second portion 472 extends to the folded, flex circuit stack 466 disposed underneath the magnetic tracking sensor assembly 404 (rather than between the ultrasound transducer 402 and the magnetic tracking sensor assembly 404). The flex circuit stack 466 includes a third portion 474 ending from the second portion 472 and folded generally perpendicularly back toward the magnetic tracking sensor assembly 404. The flex circuit stack 466 can include additional layers folded on top of the third portion 474.

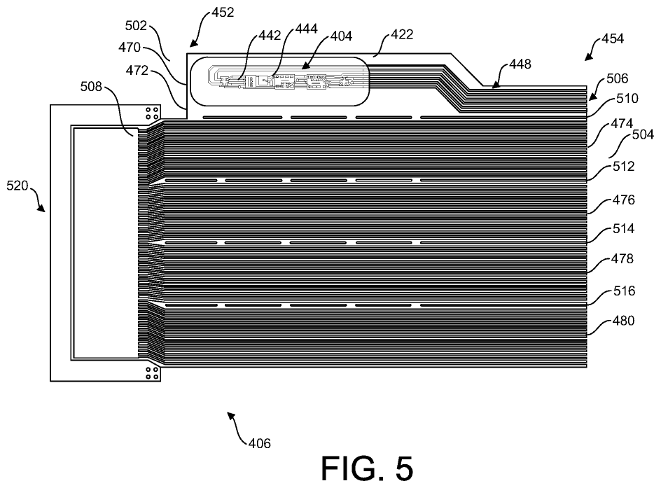

FIG. 5 illustrates a section of the example flex circuit 406 after the magnetic tracking sensor assembly 404 has been electrically coupled to mounting pads 444 and covered with the epoxy encapsulant 424 but prior to folding. The flex circuit 406 includes a distal portion 502, which can be extended to the distal end of the distal tip cover 450, and a proximal portion 504, which can be disposed proximate the catheter shaft 208. The flex circuit 406 includes the plurality of conductive elements 448, such as a generally longitudinally extending first set of conductive elements 506 associated with, and electrically coupled with, the magnetic tracking sensor assembly 404 and a generally longitudinally extending second set of conductive elements 508 associated with, and to be electrically coupled with, the electrical connectors 440 of the ultrasound transducer 402. The flex circuit 406 includes the first portion 470 including the magnetic tracking sensor assembly 404 electrically coupled to mounting pads 444 covered with the epoxy encapsulant 424 on the second side 422. The second portion 472 is adjacent the first portion 470. The interface between the second portion 472 and a remaining third portion 474 includes a longitudinally extending first perforation set 510. Similarly, the interfaces between additional remaining portions 476, 478, 480, which can form additional layers of the flex circuit stack 466, can include additional sets of perforations 512, 514, 516. During manufacture, the flex circuit 406 can be longitudinally folded along the sets of perforations 510-516 to create the flex circuit stack 466. The example flex circuit 406 also includes a distal region 520 including the distal ends of the second set of conductive elements 508, which can be laterally folded back toward the proximal portion 504 in a manner to present the ends of the second set of conductive elements 508 for attaching to the electrical connectors 440 of the ultrasound transducer 402.

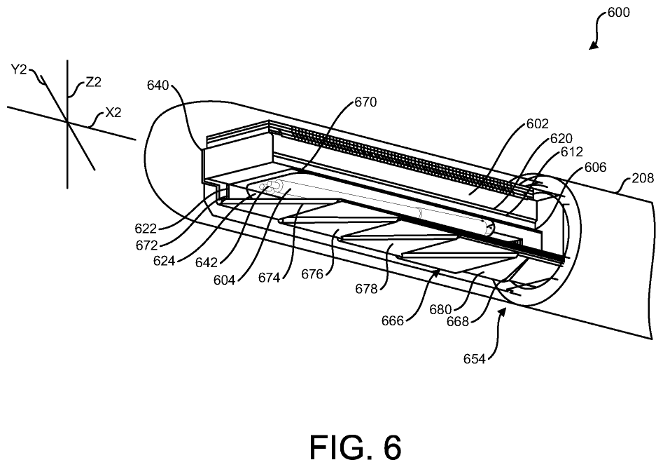

FIG. 6 illustrates an example medical imaging device 600 constructed in accordance with the medical imaging device 400. In the example, the medical imaging device 600 is installed on a medical imaging catheter 80. The medical imaging device 600 includes an ultrasound transducer 602, a magnetic tracking sensor assembly 604, and a flex circuit 606 electrically coupled to the ultrasound transducer 602 and the magnetic tracking sensor assembly 604. In the example, the medical imaging device 600 includes a longitudinal axis X2, and the longitudinal axis X2 can correspond to and be coaxial with longitudinal axis A in the case of the medical imaging device 600 disposed on the distal end region 204 of medical imaging catheter 80. In the example, the medical imaging device 600 can include, or in some examples be enclosed by, a distal tip cover 650 that is suitable for attachment to the distal end region 204 of the medical imaging catheter 80 such as abutted against the catheter shaft 208.

The ultrasound transducer 602 is formed as an acoustic stack 608 having, for example, an active layer 614, a backing layer 616, and matching layers 618. The matching layer 618 provides an active side 610 of the acoustic stack 608, and the backing layer 616 provides a backing side 612 of the acoustic stack 608. The acoustic stack 608 is developed as layers along a vertical axis Z2 generally perpendicular to the longitudinal axis X2 in a radial direction.

The magnetic tracking sensor assembly 604 of the example incorporates a plurality of inductive sensors assembled directly to the second side 622. For instance, the magnetic tracking sensor assembly 604 includes a dual-axis configuration having two inductive sensors angled away from the longitudinal axis X2 toward the proximal end 654 and converging toward the distal end 652 at a selected angle W. In one example, the inductive sensors are angled W of at least eleven degrees for effective orientation sensitivity along the longitudinal axis X2 about which the catheter shaft 208 will likely rotate in normal use and to fit on the medical imaging device 600.

The flex circuit 606 includes a first side 620 interfacing with the backing side 612 of the ultrasound transducer 602, and the flex circuit 606 includes an opposite, second side 622 interfacing with the inductive sensors of the magnetic tracking sensor assembly 604. The ultrasound transducer 602 includes electrical connectors 640 that are attached to the flex circuit on first side 620 of the flex circuit 606, and the inductive sensors of the magnetic tracking sensor assembly 604 include electrical connectors 642 that are attached to the second side 622 of the flex circuit 606. The flex circuit 606 includes a distal section 652 having a plurality of exposed electrical mounting pads 644 on a surface of the flex circuit 606. The electrical mounting pads 644 are conductively coupled via a plurality of conductive elements 648 to a plurality of electrical lead pads (not shown) on a proximal section 654 of the flex circuit 606. The magnetic tracking sensor assembly 404 is coupled to the second side 622 of the flex circuit 606 such as with electrical connectors 640 of the inductive sensors attached to associated electrical mounting pads 644. The backing side 612 of the ultrasound transducer 602 can be disposed against or attached to the first side 620 of the flex circuit 606 and the electrical connectors 640 of the ultrasound transducer 602 are electrically coupled to associated electrical mounting pads 644. In one example, the electrical connectors 640, 642 are coupled to the associated electrical connectors via wire bonds. In the example, the magnetic tacking sensor assembly 604, electrically coupled to the flex circuit 606 is covered with an epoxy encapsulant 624, disposed on the magnetic tracking sensor assembly 604 and the second side 622 of the flex circuit 606, such as for rigidity or protection of the components of the magnetic tracking sensor assembly 604.

The examples illustrate the medical imaging device 600 having a flex circuit stack 666 disposed underneath the ultrasound transducer 602 and the magnetic tracking sensor assembly 604. The flex circuit 606 is folded into the flex circuit stack 666 to include a plurality of flex circuit layers 668 developed along the vertical axis Z2. The flex circuit 606 includes a first portion 670 including the first side 620 interfacing with the backing side 612 of the ultrasound transducer 602 and the second side 622 electrically coupled to the magnetic tracking sensor assembly 604 and including the encapsulant 624. The first portion 670 of the flex circuit 606 extends to a second, or vertical portion 672 that is folded generally perpendicularly to the first portion 670 having a height along the Z2 axis sufficient to clear the magnetic tracking sensor assembly 604 or the encapsulant 624 if included. The second portion 672 is folded from the first portion 670 to fit within the electrical connectors 640, such as within the width of the backing side 612. The second portion 672 extends to the folded, flex circuit stack 666 disposed underneath the magnetic tracking sensor assembly 604 (rather than between the ultrasound transducer 602 and the magnetic tracking sensor assembly 604). The flex circuit stack 666 includes a third portion 674 ending from the second portion 672 and folded generally perpendicularly back toward the magnetic tracking sensor assembly 604. The flex circuit stack 666 can include additional layers folded on top of the third portion 674.

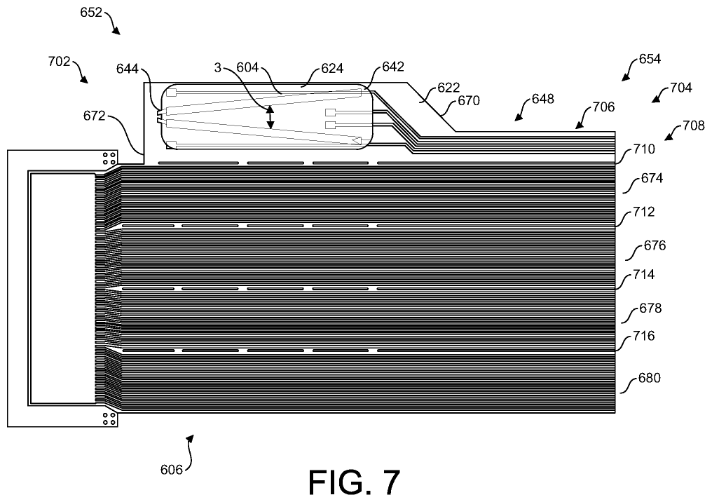

FIG. 7 illustrates a section of the example flex circuit 606 after the magnetic tracking sensor assembly 604 has been electrically coupled to mounting pads 644 and covered with the epoxy encapsulant 624 but prior to folding. The flex circuit 606 includes a distal portion 702, which can be extended to the distal end of the distal tip cover 650, and a proximal portion 704, which can be disposed proximate the catheter shaft 208. The flex circuit 606 includes the plurality of conductive elements 648, such as a generally longitudinally extending first set of conductive elements 706 associated with, and electrically coupled with, the magnetic tracking sensor assembly 604 and a generally longitudinally extending second set of conductive elements 708 associated with, and to be electrically coupled with, the electrical connectors 640 of the ultrasound transducer 602. The flex circuit 606 includes the first portion 670 including the magnetic tracking sensor assembly 604 electrically coupled to mounting pads 644 covered with the epoxy encapsulant 624 on the second side 622. The second portion 672 is adjacent the first portion 670. The interface between the second portion 672 and third portion 674 includes a longitudinally extending first perforation set 710. Similarly, the interfaces between remaining portions 676, 678, 680, which form additional layers of the flex circuit stack 666, can include additional sets of perforations 712, 714, 716. During manufacture, the flex circuit 406 can be longitudinally folded along the sets of perforations 710-716 to create the flex circuit stack 666.

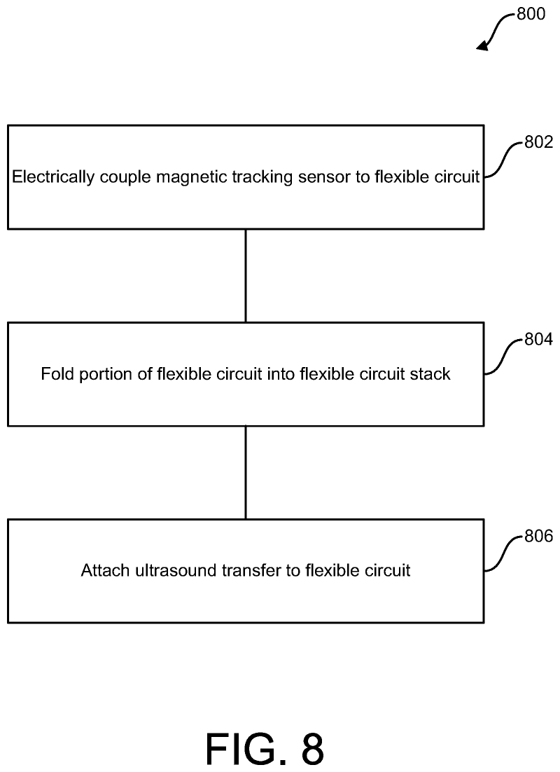

FIG. 8 illustrates an example method 800 of manufacturing the medical device 300, or medical devices 400, 600. A magnetic tracking sensor assembly is electrically coupled to a first portion of a flexible circuit, at 802. In one example, the first portion of the flexible circuit is first portion 470 and 670 on flexible circuits 406, 606, respectively. The magnetic tracking sensor assembly can be wire bonded to the exposed pads on the flexible circuit, and, in some examples, subsequently encapsulated in epoxy. A remaining portion of the flexible circuit is folded into a flexible circuit stack, at 804. For instance, the flexible circuit stack is disposed on the magnetic tracking sensor assembly, which can be encapsulated by the epoxy prior to folding. An ultrasound transducer is electrically coupled to the flexible circuit, in which the flexible circuit includes a first side of the first portion interfacing with the backing side and an opposite, second side of the first portion interfacing with the magnetic tracking sensor assembly, at 806. In one example, the ultrasound transducer is electrically coupled to the flexible circuit prior to folding the flexible circuit into the flexible circuit stack at 804. In this example, the portion of the flex circuit that extend from the longitudinal sides of the ultrasound transducer get folded prior to the ultrasound transducer being diced to separate bulk layers into individual elements. In another example, the ultrasound transducer is electrically coupled to the flexible circuit prior to the magnet tracking sensor assembly at 802.

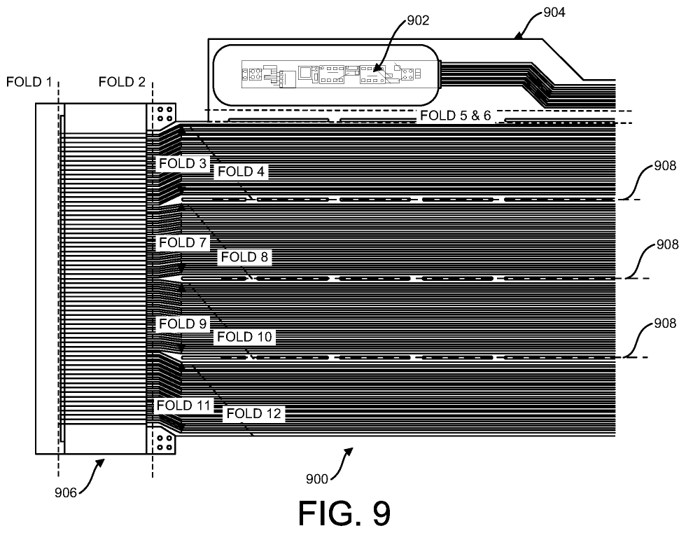

FIG. 9 illustrates an implementation of folding an assembly 900 including the flexible circuit into the flexible circuit stack, such as at 804 of method 800. The assembly 900 includes a magnetic tracking sensor assembly 902 electrically coupled to the flexible circuit 904, such as 802. The assembly further includes an ultrasound transducer 906 electrically coupled to the flexible circuit 904. In the embodiment illustrated in FIG. 9, flexible circuit 904 is cut along perforation lines 908 and folded along fold lines FOLD1 to FOLD12 to form the flexible circuit stack. For instance, the flexible circuit 904 is cut at the perforation lines 908, includes the attached magnetic tracking sensor assembly 902 and ultrasound transducer 906, and folded in order of fold lines FOLD1, then along FOLD2, then along FOLD3 . . . then along FOLD12.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical imaging device, comprising:
an ultrasound transducer having an acoustic stack comprising an active surface opposite a backing surface;
a magnetic tracking sensor assembly; and
a layered circuit assembly electrically and mechanically coupled to the ultrasound transducer and the magnetic tracking sensor assembly, the magnetic tracking sensor assembly having a coupling surface;
wherein the backing surface aligns with the coupling surface.

2. The medical imaging device of claim 1, wherein the layered circuit assembly includes a flexible circuit.

3. The medical imaging device of claim 2, wherein the flexible circuit includes a first portion coupled to a folded stack, wherein the magnetic tracking sensor assembly and ultrasound transducer are electrically coupled to the first portion, and the foldable stack is folded underneath the first portion.

4. The medical imaging device of claim 3, wherein the flexible circuit includes a plurality of sets of longitudinally extending conductive elements disposed between longitudinally extending perforations.

5. The medical imaging device of claim 3, wherein the ultrasound transducer and the magnetic tracking sensor assembly each include a plurality of electrical connections electrically coupled to the longitudinally extending conductive elements.

6. The medical imaging device of claim 1, wherein the magnetic tracking sensor assembly is disposed within an encapsulant.

7. The medical imaging device of claim 6, wherein the encapsulant is an epoxy.

8. The medical imaging device of claim 1, wherein the magnetic tracking sensor assembly includes one of a plurality of tunneling magneto-resistive sensors and a plurality of inductive sensors.

9. The medical imaging device of claim 8, wherein the plurality of inductive sensors each include longitudinal portion angled toward each other.

10. The medical imaging device of claim 8, wherein the magnetic tracking sensor assembly includes the plurality of tunneling magneto-resistive sensors electrically coupled to corresponding sensor circuits.

11. The medical imaging device of claim 1, wherein the ultrasound transducer includes a matching layer, wherein the matching layer includes the active surface.

12. The medical imaging device of claim 1, wherein the ultrasound transducer is configured as a two-dimensional imaging phased array assembly, which include a one-dimensional device.

13. The medical imaging device of claim 1, wherein the ultrasound transducer includes a backing layer, the backing layer having the backing surface, wherein the backing surface includes a plurality of grooves in the backing layer.

14. A medical imaging system, comprising:
a magnetic field transmitter assembly configured to generate a magnetic field;
a medical imaging catheter having a distal end region including a medical imaging module, the medical imaging module comprising:
an ultrasound transducer having an acoustic stack comprising an active surface opposite a backing surface, the ultrasound transducer configured to transform acoustic energy received by the acoustic stack and to provide a corresponding sensing signal;
a magnetic tracking sensor assembly, the magnetic tracking assembly configured to sense the generated magnetic field and to provide a corresponding tracking signal associated with a location of the magnetic tracking sensor assembly; and
a layered circuit assembly electrically and mechanically coupled to the ultrasound transducer and the magnetic tracking sensor assembly, the magnetic tracking sensor assembly having a coupling surface;
wherein the backing surface aligns with the coupling surface; and
a controller operably coupled to the medical imaging module, the controller configured to receive the sensing signal and the tracking signal and to generate an image including location of two-dimensional imaging plane in a three-dimensional space.

15. The medical imaging system of claim 14, wherein the controller is further configured to generate a three-dimensional map of the heart.

16. The medical imaging system of claim 14, wherein the tracking signal further provides orientation of the magnetic tracking sensor assembly and the generated image includes the location and an orientation of the two-dimensional imaging plane.

17. The medical imaging device of claim 14, wherein the ultrasound transducer is configured as a two-dimensional imaging phased array assembly having a one-dimensional array device to provide two-dimensional images by steering an acoustic beam across a two-dimensional plane.

18. A method for manufacturing a medical imaging device, the method comprising:
electrically coupling a magnetic tracking sensor assembly to a first portion of a flexible circuit;
folding a remaining portion of the flexible circuit into a flexible circuit stack; and
electrically coupling an ultrasound transducer to the flexible circuit, wherein the flexible circuit includes a first side of the first portion interfacing with the backing surface and an opposite, second side of the first portion interfacing with the magnetic tracking sensor assembly.

19. The method of claim 18, wherein the flexible circuit stack is disposed on the magnetic tracking sensor assembly.

20. The method of claim 19, wherein the magnetic tracking sensor assembly is encapsulated in an epoxy prior to folding the flexible circuit into the flexible circuit stack.
